(19) United States
(12) Patent Application Publication
(10) Pub. No.: US 2021/0076920 A1
(43) Pub. Date: Mar. 18, 2021

(54) ENDOSCOPE

(71) Applicant: OLYMPUS CORPORATION, Tokyo (JP)

(72) Inventors: Takahiro INOUE, Tokyo (JP); Akihiko OZAO, Tokyo (JP)

(73) Assignee: OLYMPUS CORPORATION, Tokyo (JP)

(21) Appl. No.: 17/103,996

(22) Filed: Nov. 25, 2020

Related U.S. Application Data
(63) Continuation of application No. PCT/JP2019/005999, filed on Feb. 19, 2019.

(30) Foreign Application Priority Data
Jun. 5, 2018 (JP) 2018-107468

(51) Int. Cl. A61B 1/06; A61B 1/00; A61B 1/07

(52) U.S. Cl. CPC A61B 1/0623; A61B 1/07; A61B 1/00096

(57) ABSTRACT

An endoscope includes: an illumination optical system that transmits illumination light generated by a light source therethrough to radiate the illumination light onto a subject, the illumination optical system being made of a transparent medium; an objective optical system that collects light from the subject irradiated with the illumination light; and a distal-end frame that accommodates the illumination optical system and the objective optical system, the distal-end frame being made of a scattering medium, wherein the illumination optical system radiates one part of the illumination light that has been transmitted therethrough indirectly onto the subject through the distal-end frame and radiates an other part of the illumination light directly onto the subject without passing through the distal-end frame.

### CROSS-REFERENCE TO RELATED APPLICATIONS

This is a continuation of International Application PCT/JP2019/005999, with an international filing date of Feb. 19, 2019, which is hereby incorporated by reference herein in its entirety.

This application is based on Japanese Patent Application No. 2018-107468, the content of which is incorporated herein by reference.

### TECHNICAL FIELD

The present invention relates to an endoscope for medical use.

### BACKGROUND ART

At the time of close-up observation, there are cases in which the illumination distribution becomes uneven due to an illumination layout, making a region close to an illumination lens brightly illuminated, whereas the other regions are darkly illuminated, thereby making it difficult to perform observation. There are known technologies for solving such unevenness of the illumination distribution, i.e., parallax (for example, see PTLs 1, 2, and 3).

The technology described in PTL 1 realizes an illumination distribution that is biased toward an observation optical system by providing a reflecting surface on a section of an illumination lens. In the technology described in PTL 2, at a distal end of an insertion portion of an endoscope, illumination light generated by a light source is guided in the circumferential direction by a ring-shaped light guide part and is scattered by a scattering part, thereby reducing the bias of the positional distribution at a light emitting part. The technology described in PTL 3 uses a liquid crystal lens or a liquid crystal prism as an illumination lens to change the focal distance, thereby changing the light distribution in accordance with the observation distance.

### CITATION LIST

### Patent Literature

{PTL 1} Japanese Unexamined Patent Application, Publication No. Sho 58-066910

{PTL 2} Publication of Japanese Patent No. 5526011

{PTL 3} Japanese Unexamined Patent Application, Publication No. Hei 02-148013

### SUMMARY OF INVENTION

One aspect of the present invention is directed to an endoscope including: an illumination optical system that transmits illumination light generated by a light source therethrough to radiate the illumination light onto a subject, the illumination optical system being made of a transparent medium; an objective optical system that collects light from the subject irradiated with the illumination light; and a distal-end frame that accommodates the illumination optical system and the objective optical system, the distal-end frame being made of a scattering medium, wherein the illumination optical system radiates one part of the illumination light that has been transmitted therethrough indirectly onto the subject through the distal-end frame and radiates an other part of the illumination light directly onto the subject without passing through the distal-end frame.

Another aspect of the present invention is directed to an illumination unit including: an illumination optical system that transmits illumination light generated by a light source therethrough to radiate the illumination light onto a subject, the illumination optical system being made of a transparent medium; and a distal-end frame that accommodates the illumination optical system, the distal-end frame being made of a scattering medium, wherein the illumination optical system radiates one part of the illumination light that has been transmitted therethrough indirectly onto the subject through the distal-end frame and radiates an other part of the illumination light directly onto the subject without passing through the distal-end frame.

### BRIEF DESCRIPTION OF DRAWINGS

FIG. 1 is an external view of a distal-end frame of an endoscope according to a first embodiment of the present invention.

FIG. 2 is a longitudinal sectional view of the distal-end frame, showing an illumination optical system and an objective optical system in the distal-end frame shown in FIG. 1.

FIG. 3 is a view showing the states of a probability density function P(g,θ) when an anisotropic scattering coefficient g=−0.3, 0, 0.3, and 0.5.

FIG. 4 is a graph for explaining the dependence of the rate of forward scattering upon the anisotropic scattering coefficient g.

FIG. 5 is a view for explaining illumination performed by a conventional endoscope serving as a Comparative Example of the endoscope according to this embodiment.

FIG. 6 is an external view of an illumination optical system according to an Example of the first embodiment.

FIG. 7 is a longitudinal sectional view of a distal-end frame of an endoscope according to a second embodiment of the present invention, showing an illumination optical system and an objective optical system in the distal-end frame.

FIG. 8 is an external view of the illumination optical system shown in FIG. 7.

FIG. 9 is another longitudinal sectional view of the distal-end frame shown in FIG. 7.

FIG. 10 is an external view of an illumination optical system according to Example 1 of the second embodiment.

FIG. 11 is an external view of an illumination optical system according to Example 2 of the second embodiment.

FIG. 12 is an external view of an illumination optical system according to Example 3 of the second embodiment.

FIG. 13 is an external view of an illumination optical system according to Example 4 of the second embodiment.

FIG. 14 is an external view of an illumination optical system according to Example 5 of the second embodiment.

FIG. 15 is an external view of an illumination optical system according to Example 6 of the second embodiment.

FIG. 16 is an external view of an illumination optical system according to Example 7 of the second embodiment.

FIG. 17 is an external view of an illumination optical system according to Example 8 of the second embodiment.

### DESCRIPTION OF EMBODIMENTS

### First Embodiment

An endoscope according to a first embodiment of the present invention will be described below with reference to the drawings.

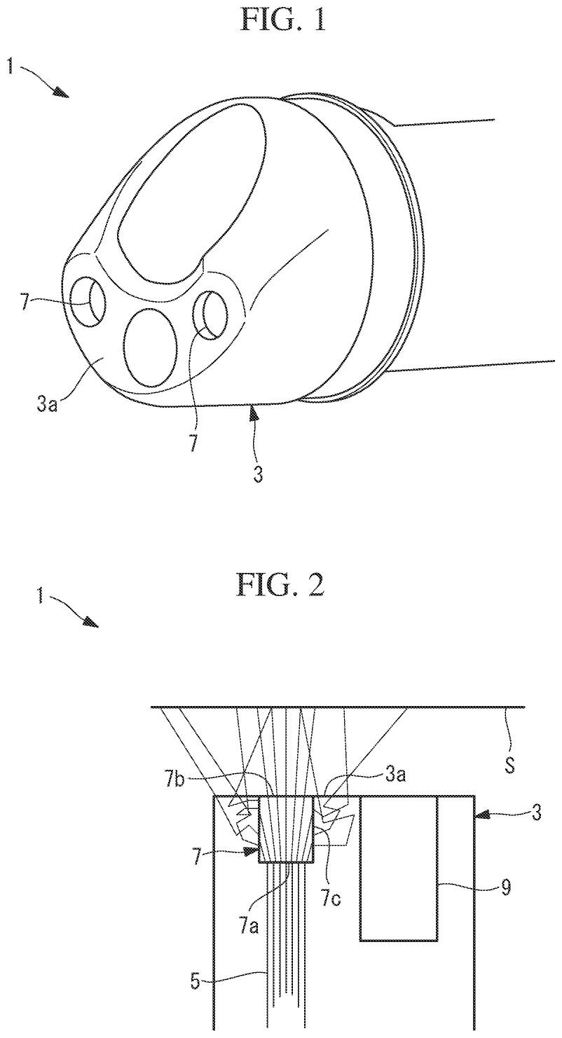

As shown in FIG. 1, an endoscope 1 of this embodiment includes a distal-end frame 3 that is disposed at a distal end of an elongated insertion portion (not shown) to be inserted into a body cavity.

The distal-end frame 3 is made of a scattering medium. As shown in FIGS. 1 and 2, the distal-end frame 3 accommodates: a section of a light guide fiber 5 that guides illumination light generated by a light source (not shown) to the distal-end frame 3; two illumination optical systems 7 that radiate the illumination light guided by the light guide fiber 5 onto a subject S; and an objective optical system 9 that collects light from the subject S irradiated with the illumination light.

The light guide fiber 5 is provided inside the insertion portion along the longitudinal direction of the insertion portion, and a distal-end section thereof is disposed inside the distal-end frame 3. The light guide fiber 5 emits, from a distal end thereof, illumination light that has entered from a base end thereof to make the emitted illumination light enter the respective illumination optical systems 7.

The two illumination optical systems 7 are each made of a transparent medium and are disposed with a gap therebetween in the width direction of the distal-end frame 3. The illumination optical systems 7 each have an incident surface 7a that illumination light emitted from the light guide fiber 5 enters, a distal-end surface 7b that is made to face the subject S, and a side surface 7c that is disposed between the incident surface 7a and the distal-end surface 7b.

Furthermore, the illumination optical systems 7 each have positive refractive power, and transmit, therethrough, illumination light entering from the incident surface 7a, thereby emitting the illumination light from the distal-end surface 7b and the side surface 7c. The respective illumination optical systems 7 are molded with resin, integrally with the distal-end frame 3, so that illumination light emitted from the entire side surfaces 7c of the respective illumination optical systems 7 enters the distal-end frame 3 without being blocked. The distal-end surfaces 7b of the respective illumination optical systems 7 are exposed at a distal-end surface (emission surface) 3a of the distal-end frame 3 that is made to face the subject S.

Each of the illumination optical systems 7 allows part of the illumination light that has been transmitted therethrough to be emitted from the side surface 7c and to enter the distal-end frame 3, thereby radiating the part of the illumination light that has been emitted from the side surface 7c, indirectly onto the subject S from the distal-end surface 3a of the distal-end frame 3 through the distal-end frame 3. Each of the illumination optical systems 7 allows the other part of the illumination light that has been transmitted therethrough to be emitted from the distal-end surface 7b, thereby radiating the other part of the illumination light that has been emitted from the distal-end surface 7b, directly onto the subject S without passing through the distal-end frame 3 .

When the anisotropic scattering coefficient of the distal-end frame 3 is indicated by g, the scattering coefficient of the distal-end frame 3 is indicated by μₛ, and the distance from the incident surface 7a of the illumination optical system 7 to the distal-end surface 3a of the distal-end frame 3 is indicated by L, the following conditional expression (1) may be satisfied.

1<Lμₛ(1−g) (1)

The mean free path of light rays that travel in a straight line inside the distal-end frame 3, which is made of the scattering medium, i.e., the distance l* in which illumination light can travel in a straight line inside the distal-end frame 3 without being scattered, is expressed by the following expression.

l*=1/[μs(1−g)]

By the time illumination light that has entered the distal-end frame 3 from the side surface 7c of the illumination optical system 7 is emitted from the distal-end surface 3a of the distal-end frame 3, the number of times the illumination light is subjected to scattering in the distal-end frame 3 is L/l*.

Therefore, in order to scatter the illumination light that has entered the distal-end frame 3 one time in the distal-end frame 3, it is necessary to satisfy the following conditional expression.

(L/l*)>1

i.e.,

1<Lμₛ(1−g) (1)

If the conditional expression (1) is satisfied, it is possible to scatter the illumination light one or more times in the distal-end frame 3 and to radiate the illumination light onto the subject S over a wide light-distribution angle including a section of the distal-end frame 3.

The following conditional expression (2) may be satisfied.

0.5<P_{f}(g)^{Lμs(1-g)} (2)

Here, P_{f}(g) is expressed by the following expression and means the probability that, after the illumination light is scattered in the distal-end frame 3, the illumination light is emitted forward, i.e., toward the subject S, which the distal-end surface 3a faces, from the distal-end surface 3a of the distal-end frame 3.

$${P}_{f} ( g ) = {\int }_{- \frac{π}{2}}^{\frac{π}{2}} P ( g , θ ) d θ$$

P(g,θ) is a probability density function in which the emission angle of the illumination light that has been scattered in the distal-end frame 3, which has the anisotropic scattering coefficient g, becomes θ.

Scattering of illumination light that travels in a straight line inside the distal-end frame 3, which is made of the scattering medium, is expressed by a probability density function P(θ) by using an angle θ in the travel direction before and after scattering. The probability density function P(g,θ) is approximately expressed by the following expression by using the Henyey-Greenstein function.

$$P ( g , θ ) = \frac{1}{4 π} \frac{1 - {g}^{2}}{{( 1 + {g}^{2} - 2 g cos θ )}^{3 / 2}}$$

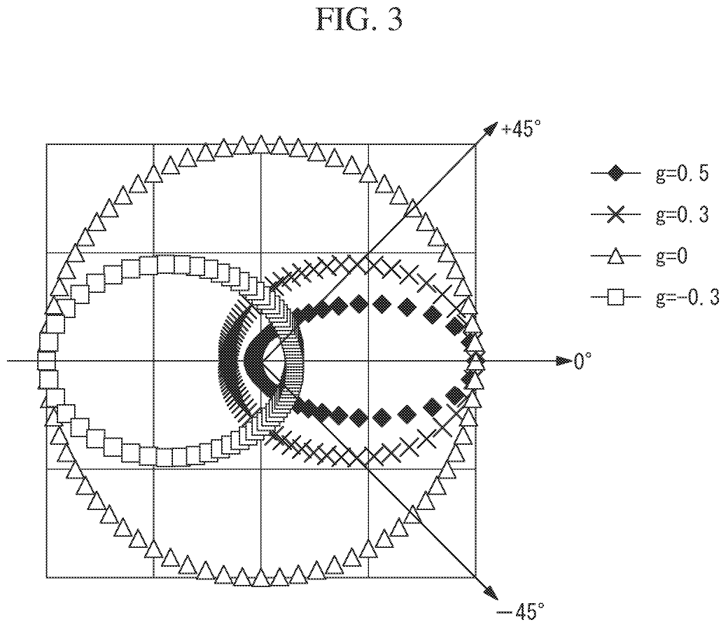

FIG. 3 shows the states of the probability density function P(g,θ) when the anisotropic scattering coefficient g=−0.3, 0, 0.3, and 0.5. When the anisotropic scattering coefficient g=0.5, illumination light is mostly scattered forward from the distal-end surface 3a of the distal-end frame 3; however, back-scattering of illumination light increases as the value of the anisotropic scattering coefficient g is reduced, thus reducing the number of components that can contribute to illumination of the subject S.

FIG. 4 shows the dependence of the rate of forward scattering upon the anisotropic scattering coefficient g. In FIG. 4, the vertical axis shows the value of P_{f}(g), and the horizontal axis shows the value of the anisotropic scattering coefficient g. From the approximate curve, the rate P_{f}(g) of illumination light that is scattered forward is simply expressed by the following expression:

P_{f}(g)=−0.51g²+1.03g+0.49

Because the number of times illumination light that travels inside the distal-end frame 3 is subjected to scattering is expressed by L*(μₛ*(1−g)), the rate of illumination light that is emitted forward from the distal-end frame 3 after repeated scattering is approximately expressed by the following expression (3):

P_{f}(g)^{Lμs(1-g)} (3)

Because at least 50% of illumination light that has entered the distal-end frame 3, which is made of the scattering medium, needs to contribute to illumination, it is preferred that the value of the conditional expression (3) be 0.5 or greater. If the value of the conditional expression (3) is 0.5 or greater, the probability that illumination light that has entered the distal-end frame 3 is scattered forward increases, thus making it possible to efficiently emit the scattered light toward the subject S.

Next, the operation of the endoscope 1 of this embodiment will be described below.

In order to observe the subject S by using the endoscope 1, which has the above-described configuration, in a state in which the insertion portion has been inserted into a body cavity, the distal-end surface 3a of the distal-end frame 3 is disposed so as to face the subject S, and illumination light is generated by the light source.

The illumination light generated by the light source is guided to the distal-end frame 3 by the light guide fiber 5 and is incident on the incident surface 7a of the illumination optical system 7. The illumination light that has been incident on the incident surface 7a is transmitted through the illumination optical system 7; then, part of the illumination light is emitted from the distal-end surface 7b toward the front side of the distal-end surface 3a, and the other part of the illumination light is emitted from the side surface 7c to enter the distal-end frame 3.

The illumination light that has been emitted from the distal-end surface 7b of the illumination optical system 7 is directly radiated onto the subject S, and the illumination light that has entered the distal-end frame 3 from the side surface 7c of the illumination optical system 7 is repeatedly scattered in the distal-end frame 3, which is made of the scattering medium, is then emitted from the distal-end surface 3a of the distal-end frame 3, and is indirectly radiated onto the subject S.

In this case, with the illumination optical system 7, part of the illumination light generated by the light source is made to pass through the distal-end frame 3 and is indirectly radiated onto the subject S from the distal-end surface 3a, thereby making it possible to radiate the illumination light onto the subject S not only from the illumination optical system 7 but also over a wide light-distribution angle including a section of the distal-end frame 3, and to improve the nonuniformity of object-surface illuminance at the time of close-up observation. Since the distal-end frame 3 has a scattering function, it is not necessary to separately provide, in the distal-end frame 3, a member for scattering illumination light. With the illumination optical system 7, the other part of the illumination light generated by the light source is directly radiated onto the subject S from the illumination optical system 7 without passing through the distal-end frame 3, thereby making it possible to suppress excessive loss of illumination light due to scattering.

According to the endoscope 1 of this embodiment, it is possible to realize an optimal illumination distribution at the time of close-up observation and at the time of non-close-up observation, while achieving a reduction in parallax at the time of close-up observation and a reduction in the diameter of the distal-end section of the endoscope.

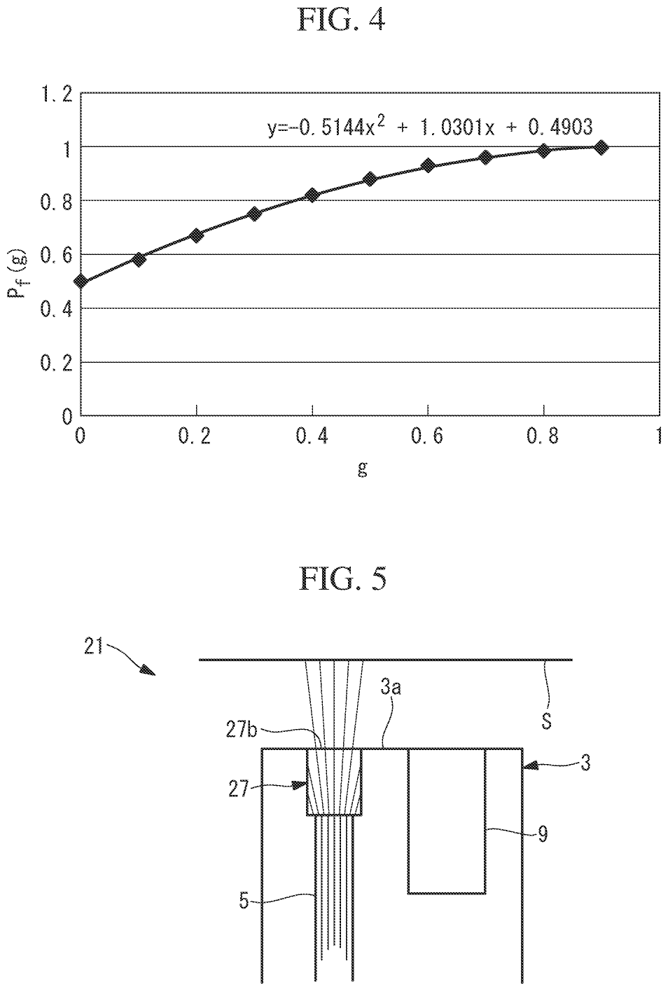

FIG. 5 shows an example conventional endoscope as a Comparative Example of the endoscope 1 of this embodiment. As shown in FIG. 5, in a conventional endoscope 21, illumination light from a light source is all directly radiated onto the subject S from a distal-end surface 27b of an illumination optical system 27. Because the part that contributes to illumination for the subject S is only the distal-end surface 27b of the illumination optical system 27, the object-surface illuminance is nonuniform at the time of close-up observation.

In this embodiment, although the illumination optical system 7 has positive refractive power, instead of this, the illumination optical system 7 may have negative refractive power. It is also possible to adopt, as the illumination optical system 7, a parallel plate that does not have refractive power.

In this embodiment, it is also possible to provide a sleeve (not shown) in the illumination optical system 7 and to accommodate the light guide fiber 5 in the sleeve, thereby holding a side surface of the light guide fiber 5. With this configuration, it is possible to easily position the light guide fiber 5.

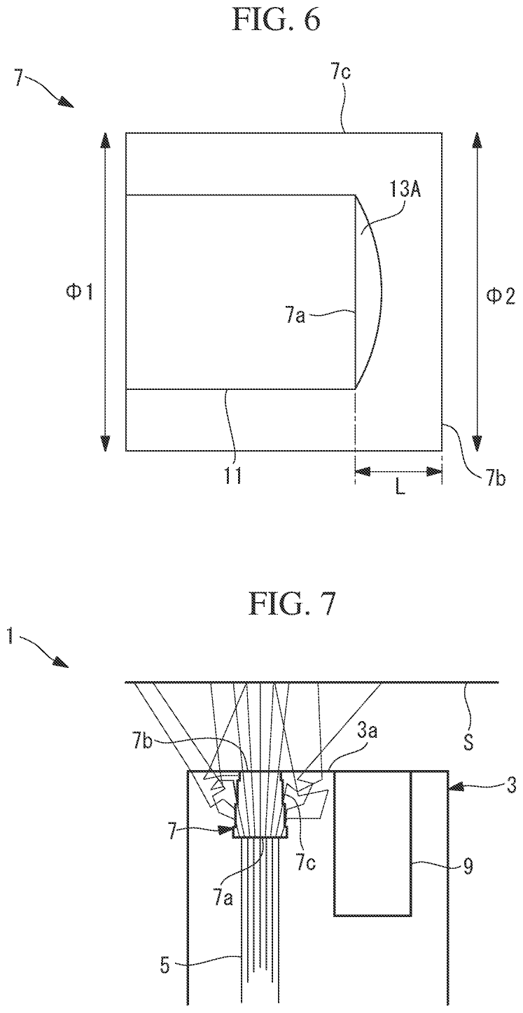

An Example of the endoscope 1 of this embodiment will be described below by using FIG. 6.

FIG. 6 is an external view of an illumination optical system 7 that is made of a transparent medium, according to this Example. A plano-concave-shaped illumination lens 13A that has a concave shape at a section thereof close to the base end of the distal-end frame 3 is formed in the illumination optical system 7. In the illumination optical system 7, the diameter Φ₁ of an end face thereof close to the base end is equal to the diameter Φ₂ of the distal-end surface 7b, and the side surface 7c has no inclination. The distance L from the incident surface 7a of the illumination optical system 7 to the distal-end surface 3a of the distal-end frame 3 is 0.150 mm, Lμₛ(1−g) is 2.4, and P_{f}^{Lμ}^{ₛ}^{(1-g)} is 0.82. In the example shown in FIG. 6, a sleeve 11 is provided, and the light guide fiber 5 is accommodated in the sleeve 11.

### Second Embodiment

Next, an endoscope according to a second embodiment of the present invention will be described below with reference to the drawings.

As shown in FIG. 7, the endoscope 1 of this embodiment differs from that of the first embodiment in terms of the shape of the illumination optical system 7.

In the description of this embodiment, identical reference signs are assigned to parts common to those of the endoscope 1 of the above-described first embodiment, and a description thereof will be omitted.

In the illumination optical system 7 of this embodiment, the diameter of the distal-end surface 7b is less than the diameter of the incident surface 7a, and the side surface 7c is formed in a tapered shape so as to become narrower from the incident surface 7a toward the distal-end surface 7b.

With this configuration, because the illumination optical system 7 is reduced in diameter toward the distal-end surface 7b, illumination light that has entered from the incident surface 7a can be more effectively transmitted to the distal-end frame 3 through the side surface 7c. Accordingly, it is easier to improve the nonuniformity of object-surface illuminance at the time of close-up observation.

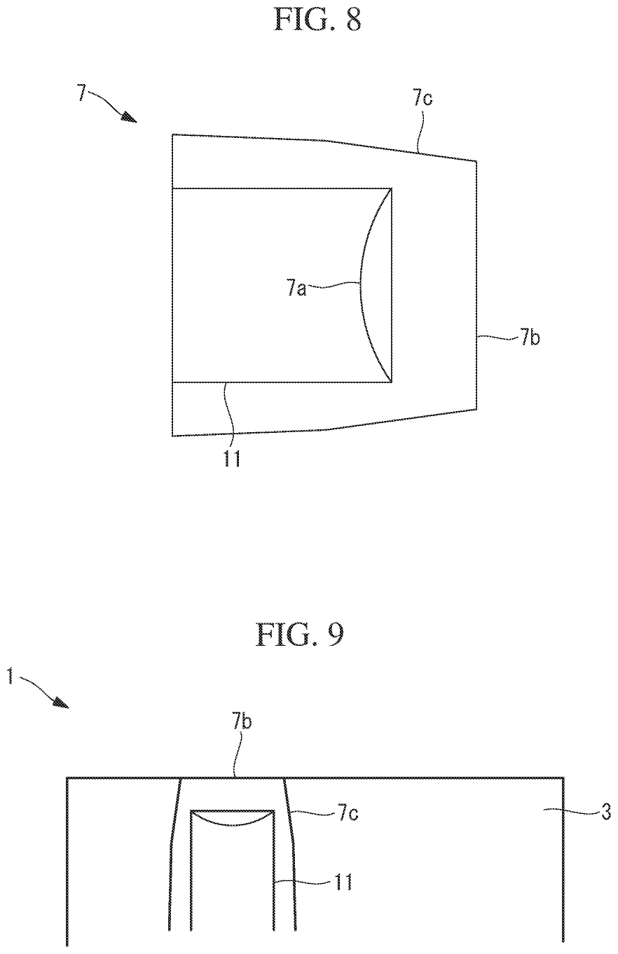

In this embodiment, for example, as shown in FIGS. 8 and 9, it is also possible to provide the sleeve 11 in the illumination optical system 7 and to accommodate the light guide fiber 5 in the sleeve 11, thereby holding the side surface of the light guide fiber 5.

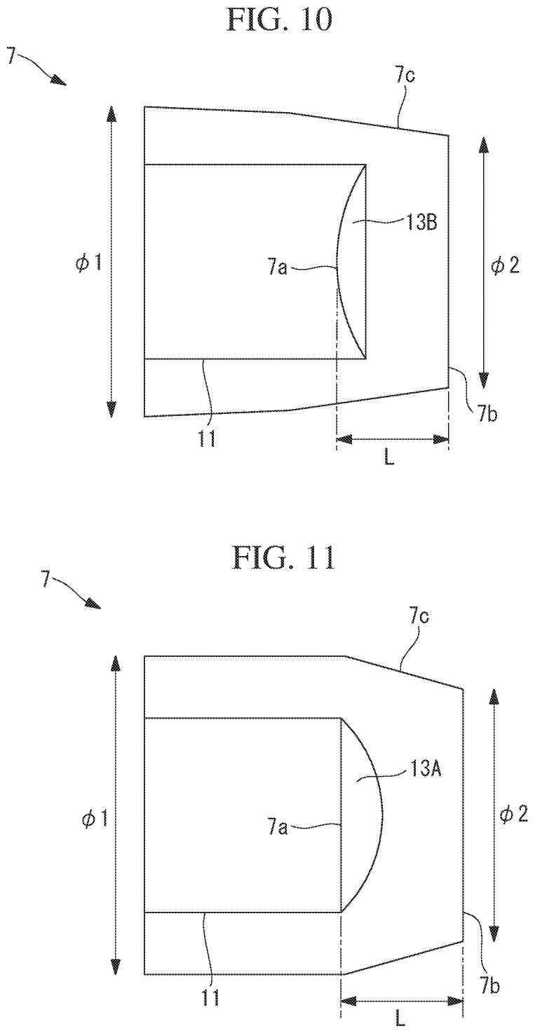

Examples 1 to 8 of the endoscope 1 of this embodiment will be described below by using FIGS. 10 to 17.

### EXAMPLE 1

FIG. 10 is an external view of an illumination optical system 7 that is made of a transparent medium, according to Example 1. A plano-convex-shaped illumination lens 13B that has a convex shape at a section thereof close to the base end of the distal-end frame 3 is formed in the illumination optical system 7. In the illumination optical system 7, the diameter Φ₂ of the distal-end surface 7b is less than the diameter Φ₁ of an end face thereof close to the base end, and the side surface 7c is formed in a tapered shape so as to become narrower from the incident surface 7a toward the distal-end surface 7b. The distance L from the incident surface 7a of the illumination optical system 7 to the distal-end surface 3a of the distal-end frame 3 is 0.282 mm, Lμₛ(1−g) is 4.5, and P_{f}^{Lμ}^{ₛ}^{(1-G) is} 0.69.

### EXAMPLE 2

FIG. 11 is an external view of an illumination optical system 7 that is made of a transparent medium, according to Example 2. A plano-concave-shaped illumination lens 13A that has a concave shape at a section thereof close to the base end of the distal-end frame 3 is formed in the illumination optical system 7. In the illumination optical system 7, the diameter Φ₂ of the distal-end surface 7b is less than the diameter Φ₁ of an end face thereof close to the base end, and the side surface 7c is formed in a tapered shape so as to become narrower from the incident surface 7a toward the distal-end surface 7b. The distance L from the incident surface 7a of the illumination optical system 7 to the distal-end surface 3a of the distal-end frame 3 is 0.200 mm, Lμₛ(1−g) is 3 .2, and P_{f}^{Lμ}^{ₛ}^{(1-g) is} 0.77.

### EXAMPLE 3

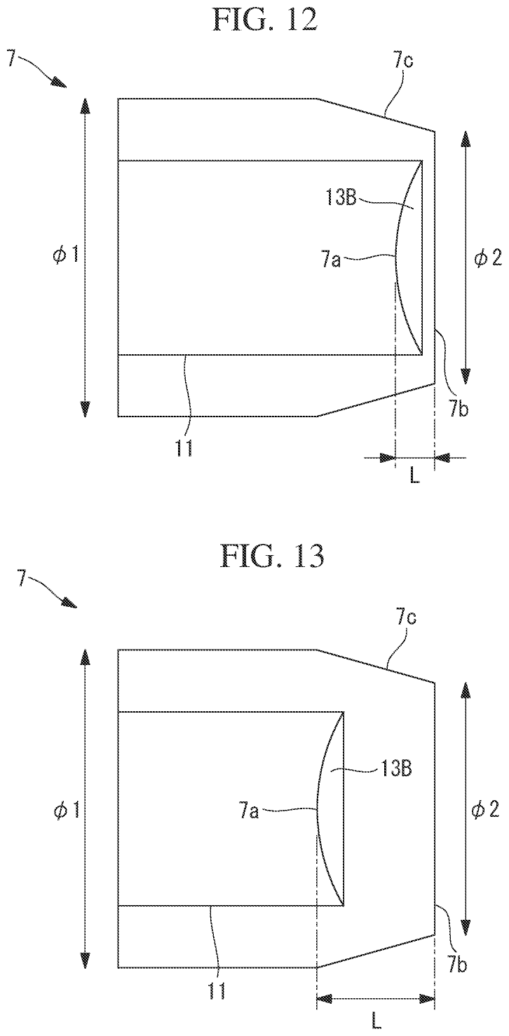

FIG. 12 is an external view of an illumination optical system 7 that is made of a transparent medium, according to Example 3 . A plano-convex-shaped illumination lens 13B that has a convex shape at a section thereof close to the base end of the distal-end frame 3 is formed in the illumination optical system 7. In the illumination optical system 7, the diameter Φ₂ of the distal-end surface 7b is less than the diameter Φ₁ of an end face thereof close to the base end, and the side surface 7c is formed in a tapered shape so as to become narrower from the incident surface 7a toward the distal-end surface 7b. Furthermore, the distance L from the incident surface 7a of the illumination optical system 7 to the distal-end surface 3a of the distal-end frame 3 is 0.100 mm, Lμₛ(1−g) is 1.6, and P_{f}^{Lμ}^{ₛ}^{(1-g)} is 0.88.

### EXAMPLE 4

FIG. 13 is an external view of an illumination optical system that is made of a transparent medium, according to Example 4. A plano-convex-shaped illumination lens 13B that has a convex shape at a section thereof close to the base end of the distal-end frame 3 is formed in the illumination optical system 7. In the illumination optical system 7, the diameter Φ₂ of the distal-end surface 7b is less than the diameter Φ₁ of an end face thereof close to the base end, and the side surface 7c is formed in a tapered shape so as to become narrower from the incident surface 7a toward the distal-end surface 7b. The distance L from the incident surface 7a of the illumination optical system 7 to the distal-end surface 3a of the distal-end frame 3 is 0.300 mm, Lμₛ(1−g) is 4.8, and P_{f}^{Lμ}^{ₛ}^{(1-g)} is 0.67.

### EXAMPLE 5

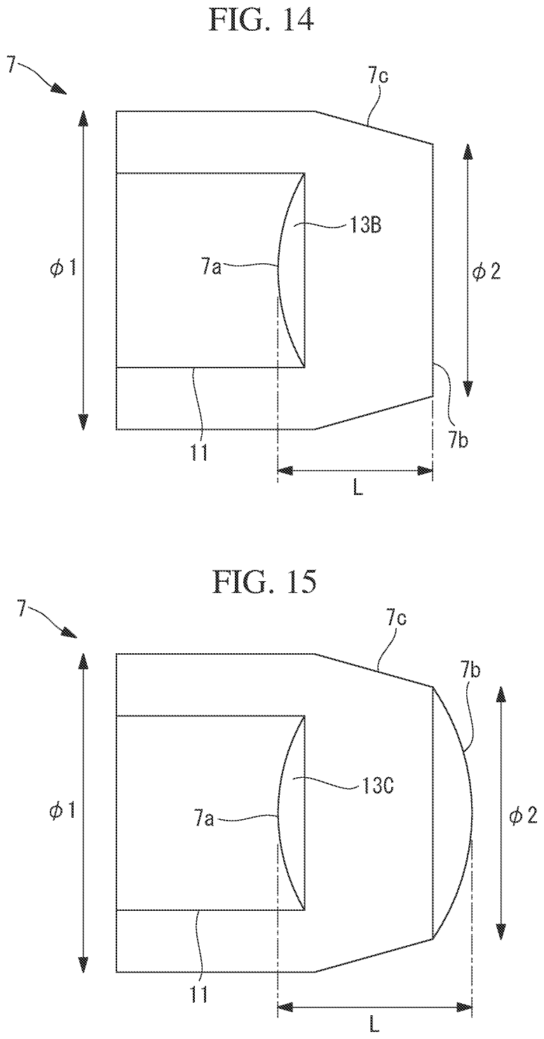

FIG. 14 is an external view of an illumination optical system that is made of a transparent medium, according to Example 5. A plano-convex-shaped illumination lens 13B that has a convex shape at a section thereof close to the base end of the distal-end frame 3 is formed in the illumination optical system 7. In the illumination optical system 7, the diameter Φ₂ of the distal-end surface 7b is less than the diameter Φ₁ of an end face thereof close to the base end, and the side surface 7c is formed in a tapered shape so as to become narrower from the incident surface 7a toward the distal-end surface 7b. The distance L from the incident surface 7a of the illumination optical system 7 to the distal-end surface 3a of the distal-end frame 3 is 0.400 mm, Lμₛ(1−g) is 6.4, and P_{f}^{Lμ}^{ₛ}^{(1-g)} is 0.59.

### EXAMPLE 6

FIG. 15 is an external view of an illumination optical system that is made of a transparent medium, according to Example 6. A biconvex-shaped illumination lens 13C that has a convex shape at a section thereof close to the base end of the distal-end frame 3 and at a section thereof close to the distal end of the distal-end frame 3 is formed in the illumination optical system 7. In the illumination optical system 7, the diameter Φ₂ of the distal-end surface 7b is less than the diameter Φ₁ of an end face thereof close to the base end, and the side surface 7c is formed in a tapered shape so as to become narrower from the incident surface 7a toward the distal-end surface 7b. The distance L from the incident surface 7a of the illumination optical system 7 to the distal-end surface 3a of the distal-end frame 3 is 0.500 mm, Lμₛ(1−g) is 8.0, and P_{f}^{Lμ}^{ₛ}^{(1-g)} is 0.52.

### EXAMPLE 7

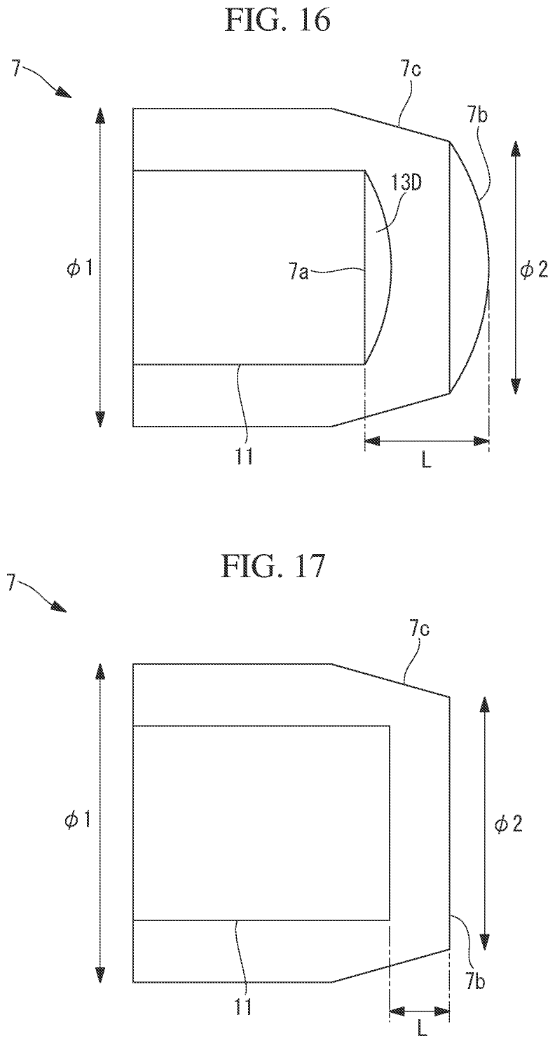

FIG. 16 is an external view of an illumination optical system that is made of a transparent medium, according to Example 7. A meniscus-shaped illumination lens 13D that has a concave shape at a section thereof close to the base end of the distal-end frame 3 and a convex shape at a section thereof close to the distal end of the distal-end frame 3 is formed in the illumination optical system 7. In the illumination optical system 7, the diameter Φ₂ of the distal-end surface 7b is less than the diameter Φ₁ of an end face thereof close to the base end, and the side surface 7c is formed in a tapered shape so as to become narrower from the incident surface 7a toward the distal-end surface 7b. The distance L from the incident surface 7a of the illumination optical system 7 to the distal-end surface 3a of the distal-end frame 3 is 0.250 mm, Lμₛ(1−g) is 4.0, and P_{f}^{Lμ}^{ₛ}^{(1-g)} is 0.72.

### EXAMPLE 8

FIG. 17 is an external view of an illumination optical system that is made of a transparent medium, according to Example 8. An optical surface that has refractive power is not formed in the illumination optical system 7. In the illumination optical system 7, the diameter Φ₂ of the distal-end surface 7b is less than the diameter Φ₁ of an end face thereof close to the base end, and the side surface 7c is formed in a tapered shape so as to become narrower from the incident surface 7a toward the distal-end surface 7b. The distance L from the incident surface 7a of the illumination optical system 7 to the distal-end surface 3a of the distal-end frame 3 is 0.150 mm, Lμₛ(1−g) is 2.4, and P_{f}^{Lμ}^{ₛ}^{(1-g)} is 0.82.

Although the embodiments of the present invention have been described in detail above with reference to the drawings, the specific configurations are not limited to those embodiments, and design changes etc. that do not depart from the scope of the present invention are also encompassed. For example, the present invention is not limited to those applied to the above-described embodiments and modifications, can be applied to an embodiment obtained by appropriately combining the above-described embodiments and modifications, and is not particularly limited.

According to one aspect, the present invention provides an endoscope including: an illumination optical system that is made of a transparent medium, that transmits illumination light generated by a light source therethrough, and that radiates the illumination light onto a subject; an objective optical system that collects light from the subject irradiated with the illumination light; and a distal-end frame that is made of a scattering medium and that accommodates the illumination optical system and the objective optical system, wherein the illumination optical system radiates part of the illumination light that has been transmitted therethrough indirectly onto the subject through the distal-end frame and radiates the other part of the illumination light directly onto the subject without passing through the distal-end frame.

According to this aspect, with the illumination optical system, part of the illumination light generated by the light source is emitted from the distal-end frame in a state in which the part of the illumination light has been scattered in the distal-end frame, which is made of the scattering medium. Accordingly, the illumination light is radiated onto the subject not only from the illumination optical system but also over a wide light-distribution angle including a section of the distal-end frame, thus making it possible to improve the nonuniformity of object-surface illuminance at the time of close-up observation. Since the distal-end frame is made to have a scattering function, it is not necessary to separately provide, in the distal-end frame, a member for scattering illumination light. With the illumination optical system, the other part of the illumination light generated by the light source is directly radiated onto the subject without passing through the distal-end frame, thereby making it possible to suppress excessive loss of the illumination light due to scattering.

Therefore, it is possible to realize an optimal illumination distribution at the time of close-up observation and at the time of non-close-up observation, while achieving a reduction in parallax at the time of close-up observation and a reduction in the diameter of the distal-end section of the endoscope.

In the above-described aspect, the illumination optical system may allow the part of the illumination light to enter the distal-end frame from a side surface disposed between an incident surface through which the illumination light enters and a distal-end surface that is made to face the subject.

With this configuration, it is possible to easily make the illumination light enter a wide area of the distal-end frame from the illumination optical system and to easily emit the illumination light from the wide area of the distal-end frame.

In the above-described aspect, the side surface may be formed in a tapered shape so as to become narrower from the incident surface toward the distal-end surface.

With this configuration, in the illumination optical system, the illumination light that has entered from the incident surface can be more effectively transmitted to the distal-end frame. Accordingly, it is possible to more easily improve the nonuniformity of object-surface illuminance at the time of close-up observation.

In the above-described aspect, when an anisotropic scattering coefficient of the distal-end frame is indicated by g, a scattering coefficient of the distal-end frame is indicated by μₛ, and the distance from the incident surface of the illumination optical system to an emission surface of the distal-end frame, from which the illumination light is emitted, is indicated by L, the following conditional expression (1) may be satisfied.

1<Lμₛ(1−g) (1)

The mean free path of light rays that travel in a straight line inside the distal-end frame, which is made of the scattering medium, i.e., the distance l* in which illumination light can travel in a straight line inside the distal-end frame without being scattered, is expressed by the following expression.

l*=1/[μs(1−g)]

Furthermore, by the time illumination light that enters the distal-end frame from the illumination optical system is emitted from the emission surface of the distal-end frame, the number of times the illumination light is subjected to scattering in the distal-end frame is L/l*.

In order to scatter the illumination light that has entered the distal-end frame one time in the distal-end frame, it is necessary to satisfy the following conditional expression.

(L/l*)>1

1<Lμₛ(1−g) (1)

If the conditional expression (1) is satisfied, it is possible to scatter the illumination light one or more times in the distal-end frame and to radiate the illumination light onto the subject over a wide light-distribution angle including a section of the distal-end frame.

In the above-described aspect, when an anisotropic scattering coefficient of the distal-end frame is indicated by g, a scattering coefficient of the distal-end frame is indicated by μₛ, and the distance from the incident surface of the illumination optical system to an emission surface of the distal-end frame, from which the illumination light is emitted, is indicated by L, the following conditional expression (2) may be satisfied:

0.5<P_{f}(g)^{Lμs(1-g)} (2)

where P_{f}(g) is expressed by the following expression and means the probability that, after the illumination light is scattered in the distal-end frame, the illumination light is emitted from the emission surface toward the subject:

$${P}_{f} ( g ) = {\int }_{- \frac{π}{2}}^{\frac{π}{2}} P ( g , θ ) d θ$$

where P(g,θ) is a probability density function in which the emission angle of the illumination light that has been scattered in the distal-end frame becomes θ.

### REFERENCE SIGNS LIST

1 endoscope

3 distal-end frame

7 illumination optical system

9 objective optical system

S subject

1. An endoscope comprising:
an illumination optical system that transmits illumination light generated by a light source therethrough to radiate the illumination light onto a subject, the illumination optical system being made of a transparent medium;
an objective optical system that collects light from the subject irradiated with the illumination light; and
a distal-end frame that accommodates the illumination optical system and the objective optical system, the distal-end frame being made of a scattering medium,
wherein the illumination optical system radiates one part of the illumination light that has been transmitted therethrough indirectly onto the subject through the distal-end frame and radiates an other part of the illumination light directly onto the subject without passing through the distal-end frame.

2. The endoscope according to claim 1, wherein the illumination optical system allows the one part of the illumination light to enter the distal-end frame from a side surface disposed between an incident surface through which the illumination light enters and a distal-end surface that is made to face the subject.

3. The endoscope according to claim 2, wherein the side surface is formed in a tapered shape so as to become narrower from the incident surface toward the distal-end surface.

4. The endoscope according to claim 2, wherein, when an anisotropic scattering coefficient of the distal-end frame is indicated by g, a scattering coefficient of the distal-end frame is indicated by μₛ, and a distance from the incident surface of the illumination optical system to an emission surface of the distal-end frame, from which the illumination light is emitted, is indicated by L, the following conditional expression (1) is satisfied.
1<Lμₛ(1−g) (1)

5. The endoscope according to claim 2, wherein, when an anisotropic scattering coefficient of the distal-end frame is indicated by g, a scattering coefficient of the distal-end frame is indicated by μₛ, and the distance from the incident surface of the illumination optical system to an emission surface of the distal-end frame, from which the illumination light is emitted, is indicated by L, the following conditional expression (2) is satisfied:
0.5<P_{f}(g)^{Lμs(1-g)} (2)
where P_{f}(g) is expressed by the following expression and means a probability that, after the illumination light is scattered in the distal-end frame, the illumination light is emitted from the emission surface toward the subject:
$${P}_{f} ( g ) = {\int }_{- \frac{π}{2}}^{\frac{π}{2}} P ( g , θ ) d θ$$
where P(g,θ) is a probability density function in which an emission angle of the illumination light that has been scattered in the distal-end frame becomes θ.

6. An illumination unit comprising:
an illumination optical system that transmits illumination light generated by a light source therethrough to radiate the illumination light onto a subject, the illumination optical system being made of a transparent medium; and
a distal-end frame that accommodates the illumination optical system, the distal-end frame being made of a scattering medium,
wherein the illumination optical system radiates one part of the illumination light that has been transmitted therethrough indirectly onto the subject through the distal-end frame and radiates an other part of the illumination light directly onto the subject without passing through the distal-end frame.
